(19) United States
(12) Patent Application Publication
(10) Pub. No.: US 2005/0267072 A1
(43) Pub. Date: Dec. 1, 2005

(54) Pharmaceutical compositions containing dually acting inhibitors of neutral endopeptidase for the treatment of sexual dysfunction

(75) Inventors: Dieter Ziegler, Hemmingen (DE); Klaus Witte, Hannover (DE); Matthias Straub, Barsinghausen (DE); Michael Weske, Burgdorf (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 11/127,162

(22) Filed: May 12, 2005

Related U.S. Application Data
(60) Provisional application No. 60/570,829, filed on May 14, 2004.

(51) Int. Cl. A61K 31/675; A61K 31/55

(52) U.S. Cl. 514/80; 514/212.07

(57) ABSTRACT

The present invention relates to the novel medicinal use of dually acting compounds capable of inhibiting neutral endopeptidase (=NEP) and human soluble endopeptidase (=hSEP) in the prophylaxis and/or treatment of sexual dysfunction in mammals and humans.

### CROSS REFERENCE TO RELATED APPLICATIONS

This application claim priority from U.S. provisional patent application No. 60/570,829, filed May 14, 2004, the entire disclosure of which is incorporated herein by reference.

### BACKGROUND OF THE INVENTION

The present invention relates to the novel medicinal use of dually acting compounds capable of inhibiting neutral endopeptidase (=NEP, E.C. 3.4.24.11) and the newly discovered human soluble endopeptidase (=hSEP) in the prophylaxis and/or treatment of sexual dysfunction in mammals and humans. In a further aspect, the invention relates to a novel pharmaceutical combination composition comprising at least one NEP inhibitor, at least one inhibitor of hSEP and at least one compound supportive to the inventive use as specified in more detail below.

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self esteem and disrupts personal relationships thereby inducing personal distress.

From document WO 99/55726 A1 it is known, that certain thiol inhibitors of ECE are useful for treating or inhibiting i.a. erectile dysfunction.

Document EP 1 097 719 A1 discloses the use of NEP inhibitors for the treatment of FSD.

In document WO 02/03995 A2, the use of NEP inhibitors or of combinations of NEP inhibitors with PDE5 inhibitors is described.

Publication WO 02/06492 A1 discloses i.a. antibodies against and inhibitors of a specific polypeptide having soluble secreted endopeptidase (=SEP) activity.

Benzazepine-, benzoxazepine- and benzothiazepine-N-acetic acid derivatives with i.a. NEP-inhibiting properties and an inhibitory effect on endothelin converting enzyme (=ECE) are known from document EP 0 733 642 A1 (=U.S. Pat. No. 5,677,297). Further favourable pharmacological properties of compounds falling within the structural scope of EP 0 733 642 A1 are known from documents EP 0 830 863 A1 (=U.S. Pat. No. 5,783,53), WO 00/48601 A1 (=U.S. Pat. No. 6,482,820) and WO 01/03699 A1 (=U.S.-2003-0040512-A1).

Phosphonic acid substituted benzazepinone-N-acidic acid derivatives with a combined inhibitory effect on NEP and ECE are disclosed in document EP 0 916 679 A1 (=U.S. Pat. No. 5,952,327).

From document WO 02/094176 A2 it is known that certain compounds, including those disclosed in document EP 0 733 642 A1 and in document EP 0 916 679 A1, may inhibit the metalloprotease enzyme IGS5. The metalloprotease IGS5 is also known as human soluble endopeptidase (=hSEP) and is described e.g. in document WO 02/094176 A2. Further, WO 02/094176 A2 discloses the use of compounds with combined NEP- and hSEP-inhibitory activity for the prophylaxis or treatment of inter alia cardiovascular diseases.

### SUMMARY OF THE INVENTION

It is the object of the present invention to provide a novel therapy for the prophylaxis and/or treatment of SD, in particular male sexual dysfunction (=MSD) in mammals and humans.

It has now surprisingly been found that a dually acting compound capable of inhibiting NEP and hSEP is particularly suitable in the prophylaxis or treatment of SD. It has further been found that a pharmaceutical composition which comprises at least one NEP inhibitor, at least one hSEP inhibitor and at least one further compound supportive to the inventive use provides specific advantages in the prophylaxis ad/or treatment of SD.

The invention therefore relates in a first aspect to the use of a dually acting compound capable of inhibiting NEP and hSEP for the prophylaxis or treatment of sexual dysfunction. More specifically, a compound of general Formula I, 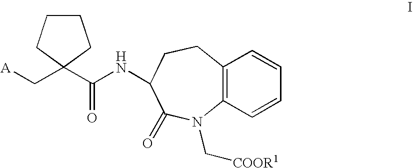
wherein
R¹ is hydrogen or a group forming a biolabile carbonic acid ester,
A represents a group selected from the subgroups a, 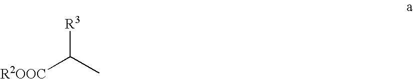
   wherein
   R² is hydrogen or a a group forming a biolabile carbonic acid ester and
   R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group or b, 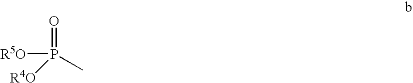
   wherein
   R⁴ is hydrogen or a group forming a biolabile phosphonic acid ester and
   R⁵ is hydrogen or a group forming a biolabile phosphonic acid ester, and/or physiologically compatible salts of acids of Formula I can be used according to the invention.

Where the substituents in the compounds of Formula I are or contain C₁₋₄-alkyl groups, these may be straight-chain or branched and contain preferably 1 to 2 carbon atoms and are preferably methyl or methoxy. Where biolabile ester forming groups in the compounds of Formula I are or contain lower alkyl groups, these may be straight-chain or branched and contain usually 1 to 4 carbon atoms. Where the substituents contain halogen, fluorine, chlorine or bromine, preferably fluorine or chlorine, are particularly suitable.

The compounds of Formula I are optionally esterified dicarboxylic acid derivatives. Depending on the form of administration, biolabile monoesters, particularly compounds in which R² is a group forming a biolabile ester and R¹ is hydrogen, or dicarboxylic acids are preferred, the latter being particularly suitable for i.v. administration.

Suitable physiologically compatible salts of dicarboxylic acids or monoesters of Formula I include their alkali metal, alkaline earth metal or ammonium salts, for example sodium or calcium salts or salts with physiologically compatible, pharmacologically neutral organic amines such as, for example, diethylamine or tert.-butylamine.

Preferred are the compounds of general Formula Ia, 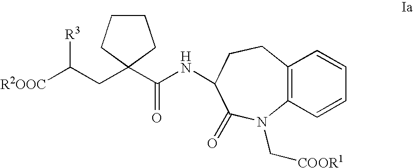
wherein R¹, R² and R³ have the above meanings, and physiologically compatible salts of acids of Formula Ia. Preferred salts of compounds of Formula Ia are e.g. disclosed in document WO 03/059939 A1. The compounds of Formula Ia contain two chiral carbon atoms, namely the carbon atom which is in the 3 position of the ring framework and bears the amide side-chain, and the carbon atom of the amide side-chain which bears the radical R³. The compounds can therefore exist in several optically active stereoisomeric forms or as a racemate. According to the present invention both the racemic mixtures and the isomerically pure compounds of Formula Ia may be used.

The compounds of Formula Ia are optionally esterified dicarboxylic acid derivatives. Depending on the form of administration, biolabile monoesters, particularly compounds in which R² is a group forming a biolabile ester and R¹ is hydrogen, or dicarboxylic acids are preferred, the latter being particularly suitable for i.v. administration. Groups which can be cleaved under physiological conditions in vivo, releasing bioavailable derivatives of the compounds of Formula Ia, are suitable as groups forming biolabile carboxylic acid esters R¹ and R². Suitable examples of this are C₁₋₄-alkyl groups, in particular methyl, ethyl, n-propyl and isopropyl; C₁₋₄-alkyloxy-C₁₋₄-alkyloxy-C₁₋₄-alkyl groups, in particular methoxyethoxymethyl; C₃₋₇-cycloalkyl groups, in particular cyclohexyl; C₃₋₇-cycloalkyl-C₁₋₄-alkyl groups, in particular cyclopropylmethyl; N,N-di-(C₀₋₄-alkyl)amino-C₁₋₆-alkyl groups; phenyl or phenyl-C₁₋₄-alkyl groups optionally substituted in the phenyl ring once or twice by halogen, C₁₋₄-alkyl or C₁₋₄-alkoxy or by a C₁₋₄-alkylene chain bonded to two adjacent carbon atoms; dioxolanylmethyl groups optionally substituted in the dioxolane ring by C₁₋₄-alkyl; C₂₋₆-alkanoyloxy-C₁₋₄-alkyl groups optionally substituted at the oxy-C₁₋₄-alkyl group by C₁₋₄-alkyl; double esters like 1-[[(C₁₋₄-alkyl)carbonyl]oxy]C₁₋₄-alkyl esters, e.g. (RS)-1-[[(isopropyl)-carbonyl]oxy]ethyl or (RS)-1-[[(ethyl)carbonyl]oxy]-2-methylpropyl (for preparation see e.g. F. W. Sum et al., Bioorg. Med. Chem. Lett. 9 (1999) 1921-1926 or Y. Yoshimura et al., The Journal of Antibiotics 39/9 (1986) 1329-1342); carbonate esters like 1-[[(C₄₋₇-cycloalkyloxy)carbonyl]oxy]C₁₋₄-alkyl esters, preferably (RS)-1-[[(cyclohexyloxy)carbonyl]oxy]ethyl (=cilexetil; for preparation see e.g. K. Kubo et al., J. Med. Chem. 36 (1993) 2343-2349, cited as “Kubo et al.” hereinafter)) or 2-oxo-1,3-dioxolan-4-yl-C₁₋₄-alkyl esters which optionally contain a double bond in the dioxolan ring, preferably 5-methyl-2-oxo-1,3-dioxolen-4-yl-methyl (=medoxomil, for preparation see e.g. Kubo et al.) or 2-oxo-1,3-dioxolan-4-yl-methyl (=(methyl)-ethylenecarbonate). Where the group forming a biolabile ester represents an optionally substituted phenyl-C₁₋₄-alkyl group, this may contain an alkylene chain with 1 to 3, preferably 1, carbon atoms and preferably stands for optionally substituted benzyl, in particular for 2-chlorobenzyl or 4-chlorobenzyl. Where the group forming a biolabile ester represents an optionally substituted phenyl group, the phenyl ring of which is substituted by a lower alkylene chain, this may contain 3 to 4, preferably 3, carbon atoms and in particular be indanyl. Where the group forming a biolabile ester represents an optionally substituted C₂₋₆-alkanoyloxy-C₁₋₄-alkyl group, the C₂₋₆-alkanoyl group may be straight-chain or branched.

R¹ preferably has the meanings hydrogen, C₁₋₄-alkyl, p-methoxybenzyl, N,N-di-(C₀₋₄-alkyl)amino-C₁₋₆-alkyl, (RS)-1-[[(isopropyl)carbonyl]oxy]ethyl, (RS)-1-[[(ethyl)carbonyl]oxy]-2-methylpropyl, (RS)-1-[[(cyclohexyloxy)carbonyl]oxy]ethyl, 5-methyl-2-oxo-1,3-dioxolen-4-yl-methyl, 2-oxo-1,3-dioxolan-4-yl-methyl or (RS)-1-[[(ethoxy)carbonyl]oxy]ethyl.

R² preferably has the meanings hydrogen, ethyl, methoxyethoxymethyl, (RS)-1-[[(isopropyl)carbonyl]oxy]ethyl, (RS)-1-[[(ethyl)carbonyl]oxy]-2-methylpropyl, (RS)-1-[[(cyclohexyloxy)carbonyl]oxy]ethyl, 5-methyl-2-oxo-1,3-dioxolen-4-yl-methyl, 2-oxo-1,3-dioxolan-4-yl-methyl or (RS)-1-[[(ethoxy)carbonyl]oxy]ethyl.

In the radical R³ the lower alkylene chain of the phenyl-C₁₋₄-alkyl group may contain preferably 1 to 2 carbon atoms. In particular, R³ is an optionally substituted phenethyl group which can optionally be substituted one or two times by halogen, C₁₋₄-alkoxy or C₁₋₄-alkyl, or is a naphthylethyl group.

Of the compounds of Formula Ia, most preferred are the compounds which are selected from the group consisting of 2-[1-(1-carboxymethyl-2-oxo-2,3,4,5-tetrahydro-1H-benzo[b]azepin-3-ylcarbamoyl)-cyclopentylmethyl]-4-phenyl-butyric acid ethyl ester [alternative name: 3-[1-{2′-(ethoxycarbonyl)}-4′-phenylbutyl]-cyclopentan-1-carbonylamino]-2,3,4,5-tetrahydro-2-oxo-1H-1-benzazepin-1-acetic acid] of Formula II, 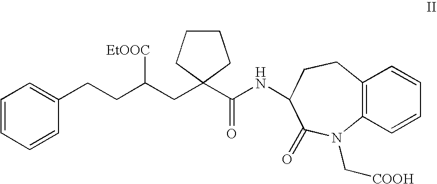
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid ethyl ester [alternative name: 3-[1-{2-(ethoxycarbonyl)-4-(1-naphthyl)butyl]cyclopentyl}carbonyl)amino]-2-oxo-2,3,4,5-tetrahydro-1H-1-benzazepin-1-yl}acetic acid] of Formula III, 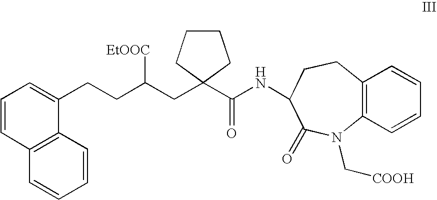
2-[1-(1-carboxymethyl-2-oxo-2,3,4,5-tetrahydro-1H-benzo[b]azepin-3-ylcarbamoyl)-cyclopentylmethyl]-4-phenyl-butyric acid of Formula IV, 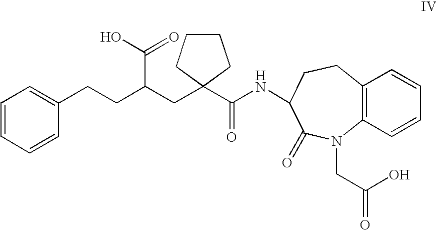
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid of Formula V, 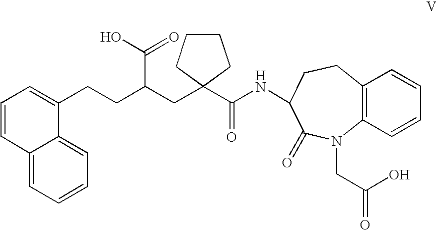
   and physiologically compatible salts of the acids of Formulas II, III, IV and/or V. The compounds of Formulas II, III, IV and V are especially suited in their 3S,2′R forms. Most preferred is the compound of Formula II in its 3S,2′R form, also known as “daglutril” or “SLV306”. The compounds of Formula Ia are known, for example, from document EP 0 733 642 A1 which is incorporated herein by reference, and can be produced according to the production processes disclosed or referenced in this document or analogously to said production processes.

Further, compounds of general Formula Ib, 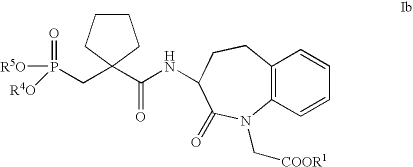
wherein R¹, R⁴ and R⁵ have the meanings given above, and physiologically compatible salts of said acids of Formula Ib can be used according to the invention. The compounds of Formula Ib are known, for example, from document EP 0 916 679 A1, and can be produced according to the production processes disclosed or referenced in this document or analogously to said production processes.

Suitable groups R¹ forming biolabile carboxylic acid esters are those as specified for compounds of Formula Ia above.

Groups R⁴ and R⁵ suitable as groups forming biolabile phosphonic acid esters are those which can be removed under physiological conditions in vivo with release of the respective phosphonic acid function. For example, groups which are suitable for this purpose are lower alkyl groups, C₂-C₆-alkanoyloxymethyl groups optionally substituted on the oxymethyl group by lower alkyl, or phenyl or phenyl-lower alkyl groups whose phenyl ring is optionally mono- or polysubstituted by lower alkyl, lower alkoxy or by a lower alkylene chain bonded to two adjacent carbon atoms. If the group R⁴ and/or R⁵ forming a biolabile ester is or contains lower alkyl, this can be branched or unbranched and can contain 1 to 4 carbon atoms. If R⁴ and/or R⁵ are an optionally substituted alkanoyloxymethyl group, it can contain a preferably branched alkanoyloxy group having 2 to 6, preferably 3 to 5, carbon atoms and can, for example, be a pivaloyloxymethyl radical (=tert-butylcarbonyloxymethyl radical). If R⁴ and/or R⁵ are an optionally substituted phenyl-lower alkyl group, this can contain an alkylene chain having 1 to 3, preferably 1, carbon atoms. If the phenyl ring is substituted by a lower alkylene chain, this can contain 3 to 4, in particular 3, carbon atoms and the substituted phenyl ring is in particular indanyl.

The compounds of the formula Ib contain a chiral carbon atom, namely the carbon atom carrying the amide side chain in the 3-position of the benzazepine structure. The compounds can thus be present in two optically active stereoisomeric forms or as a racemate. The present invention includes both the racemic mixtures and the isomerically pure compounds of the formula I. If R⁴ and R⁵ in compounds of the formula Ib are not hydrogen and in each case have different meanings, the phosphorus atom of the phosphonic acid group can also be chiral. The invention also relates to the isomer mixtures and isomerically pure compounds of the formula Ib formed as a result of chiral phosphorus atoms.

When compounds of Formula Ib are used according to the invention, (3-{[1-(benzyloxy-ethoxy-phosphorylmethyl)-cyclopentanecarbonyl]-amino}-2-oxo-2,3,4,5-tetrahydro-benzo[b]azepin-1-yl)-acetic acid tert-butyl ester and isobutyric acid 1-[[1-(-1-carboxymethyl-2-oxo-2,3,4,5-tetrahydro-1H-benzo[b]azepin-3-ylcarbamoyl)-cyclopentylmethyl]-(1-isobutyryloxy-ethoxy)-phosphinoyloxy]-ethyl ester are preferred. Both of said compounds are particularly preferred when the stereochemistry at the chiral carbon atom (see above) is “S”, namely in their “(3S)” configuration.

The compounds of the invention, in particular the compounds of Formula Ia are suited for the prophylaxis or treatment of SD. In the clinic, SD disorders have been divided into female sexual dysfunction (FSD) disorders and male sexual dysfunction (MSD) disorders (see Melman, A. & Gingell, J. C. (1999). The epidemiology and pathophysiology of erectile dysfunction. J Urology 161 : 5-11), hereinafter cited as “Melman et al. 1999”). The dually acting compounds of the invention which are capable of inhibiting NEP and hSEP are particularly beneficial for the prophylaxis and/or treatment of MSD (e.g. male erectile dysfunction-MED). A further advantage of compounds of Formula I in this indication is a certain ECE inhibitory share at their profile of action.

MSD is generally associated with erectile dysfunction, also known as male erectile dysfunction (=MED) (see Benet, A. E. et al (1994), Male erectile dysfunction assessment and treatment options. C07Sp. TheY. 20: 669-673) hereinafer cited as “Benet et al. 1994”). MED is defined as: “ . . . the inability to achieve and/or maintain a penile erection for satisfactory sexual performance (see NIH Consensus Development Panel on Impotence (1993). NIH Consensus Conference Impotence. JA. M. A. 270: 83) . . . ”. It has been estimated that the prevalence of erectile dysfunction (=ED) of all degrees (minimal, moderate and complete impotence) is 52% in men 40 to 70 years old, with higher rates in those older than 70 (Melman et al. 1999). The condition has a significant negative impact on the quality of life of the patient and their partner, often resulting in increased anxiety and tension which leads to depression and low self esteem. Whereas two decades ago, MED was primarily considered to be a psychological disorder (Benet et al. 1994), it is now known that for the majority of patients there is an underlying organic cause. As a result, much progress has been made in identifying the mechanism of normal penile erection and the pathophysiology of MED.

When the dually acting compounds capable of inhibiting NEP and hSEP of the invention are used in the therapy of FSD, therapy of female sexual arousal disorder (=FSAD) is preferred.

FSD is best defined as the difficulty or inability of a woman to find satisfaction in sexual expression. FSD is a collective term for several diverse female sexual disorders (Leiblum, S. R. (1998). Definition and classification of female sexual disorders. Int. J. Impotence Res., 10, S104-S106; Berman, J. R., Berman, L.& Goldstein, I. (1999). Female sexual dysfunction: Incidence, pathophysiology, evaluations and treatment options. Urology, 54,385-391.). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders. The categories of FSD are best defined by contrasting them to the phases of normal female sexual response: desire, arousal and orgasm (Leiblum, S. R. (1998). Definition and classification of female sexual disorders. Int. J. Impotence Res., 10, S104-S106).

Desire or libido is the drive for sexual expression. Its manifestations often include sexual thoughts either when in the company of an interested partner or when exposed to other erotic stimuli.

Arousal is the genital response to sexual stimulation, an important component of which is genital engorgement and includes increased vaginal blood flow, vaginal lubrication, elongation of the vagina and increased genital sensation/sensitivity.

Orgasm is the release of sexual tension that has culminated during arousal. hence, FSD occurs when a woman has an inadequate or unsatisfactory response in any of these phases, usually desire, arousal or orgasm.

FSD categories include hypoactive sexual desire disorder, sexual arousal disorder, orgasmic disorders and sexual pain disorders.

Although the compounds of the invention will improve the genital response to sexual stimulation (as in female sexual arousal disorder), in doing so they may also improve the associated pain, distress and discomfort associated with intercourse and so treat other female sexual disorders. Thus, in accordance with a particular aspect of the invention, there is provided use of a compound of the invention in the preparation of a medicament for the treatment or prophylaxis of hypoactive sexual desire disorder, sexual arousal disorder, orgasmic disorder and sexual pain disorder, more preferably for the treatment or prophylaxis of sexual arousal disorder, orgasmic disorder, and sexual pain disorder, and preferably in the treatment or prophylaxis of sexual arousal disorder. Hypoactive sexual desire disorder is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety.

FSAD is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants e.g. selective serotonin re-uptake inhibitors (=SSRIs) or antihypertensive agents.

Sexual pain disorders (includes dyspareunia and vaginismus) are characterised by pain resulting from penetration and may be caused by medications which reduce lubrication, endometriosis, pelvic inflammatory disease, inflammatory bowel disease or urinary tract problems. The prevalence of FSD is difficult to gauge because the term covers several types of problems, some of which are difficult to measure, and because the interest in treating FSD is relatively recent.

Many women's sexual problems are associated either directly with the female ageing process or with chronic illnesses such as diabetes and hypertension. Because FSD consists of several subtypes that express symptoms in separate phases of the sexual response cycle, there is not a single therapy.

Current treatment of FSD focuses principally on psychological or relationship issues. Treatment of FSD is gradually evolving as more clinical and basic science studies are dedicated to the investigation of this medical problem. Female sexual complaints are not all psychological in pathophysiology, especially for those individuals who may have a component of vasculogenic dysfunction (e.g. FSAD) contributing to the overall female sexual complaint. There are at present no drugs licensed for the treatment of FSD. Empirical drug therapy includes oestrogen administration (topically or as hormone replacement therapy), androgens or mood-altering drugs such as buspirone or trazodone. These treatment options are often unsatisfactory due to low efficacy or unacceptable side effects. Since interest is relatively recent in treating FSD pharmacologically, therapy consists of the following: psychological counselling, over-the-counter sexual lubricants, and investigational candidates, including drugs approved for other conditions. These medications consist of hormonal agents, either testosterone or combinations of oestrogen and testosterone and more recently vascular drugs, that have proved effective in MED. None of these agents has yet been demonstrated to be effective in treating FSD.

The Diagnostic and Statistical Manual (DSM) IV of the American Psychiatric Association defines FSAD as being: “ . . . a persistent or recurrent inability to attain or to maintain until completion of the sexual activity adequate lubrication-swelling response of sexual excitement. The disturbance must cause marked distress or interpersonal difficulty . . . . ”. The arousal response consists of vasocongestion in the pelvis, vaginal lubrication and expansion and swelling of the external genitalia. The disturbance causes marked distress and/or interpersonal difficulty. Studies investigating sexual dysfunction in couples reveals that up to 76% of women have complaints of sexual dysfunction and that 30-50% of women in the USA experience FSD (Berman, J. R., Berman, L. A., Werbin, T. J. et al., (1999). Female sexual dysfunction: Anatomy, physiology, evaluation and treatment options. Curr Opin Urology, 9,563-568). FSAD is a highly prevalent sexual disorder affecting pre-, peri- and post-menopausal (hormone replacement therapy (HRT)) women. It is associated with concomitant disorders such as depression, cardiovascular diseases, diabetes and urogenital disorders. The primary consequences of FSAD are lack of engorgement/swelling, lack of lubrication and lack of pleasurable genital sensation. The secondary consequences of FSAD are reduced sexual desire, pain during intercourse and difficulty in achieving an orgasm. It has recently been hypothesised that there is a vascular basis for at least a proportion of patients with symptoms of FSAD (Goldstein et al., Int. J. Impot. Res., 10, S84-S90, 1998) with animal data supporting this view (Park et al., Int. J. Impot. Res., 9, 27-37, 1997).

The present invention therefore in a second aspect provides a method of treating or preventing sexual dysfunction in mammals and humans comprising administering to a subject in need thereof an effective amount of a dually acting compound capable of inhibiting NEP and hSEP according to the invention. Typically, the physician will determine the actual dosage which will be most suitable for an individual patient and it will vary with the age, weight, sex and response of the particular patient. In general, a therapeutically effective daily oral or intravenous dose of the agents of the present invention is likely to range from 0.01 to 50 mg/kg body weight of the subject to be treated, preferably 0.01 to 20 mg/kg, more preferably 0.1 to 20 mg/kg. The agents of the present invention may also be administered by intravenous infusion, at a dose which is likely to range from 0.001-10 mg/kg/hr. The above dosages are exemplary of the average case. There can, of course, be individual instances where higher or lower dosage ranges are merited, and such are within the scope of this invention.

In a third aspect, the invention also provides a method of treating or preventing sexual dysfunction in mammals and humans comprising administering to a subject in need thereof therapeutically effective amounts of each of
a) at least one neutral endopeptidase inhibitor,
b) at least one inhibitor of human soluble endopeptidase and
c) at least one compound supportive to the inventive use which is selected from the group consisting of the specific compounds or subgroups of PDE5 inhibitors, NPY receptor antagonists, PDE2 inhibitors, nitric oxide donors, dopamine receptor agonists, melanocortin receptor agonists, potassium channel openers, calcium activated potassium channel openers, α1-adrenoceptor antagonists, vasoactive intestinal peptide (=VIP) receptor agonists or VIP analogues, α2-adrenoceptor antagonists, estrogens, medroxyprogesterone, medroxyprogesterone acetate, methyl testosterone hormone replacement therapy agents, testosterone, testostrone replacement agents, testosterone/oestradiol agents, estrogen agonists, serotonin receptor agonists or antagonists, prostanoid receptor agonists, purinergic receptor agonists, ECE inhibitors, endothelin receptor antagonists, NEP inhibitors and antidepressant agents.

It is known that inhibitors of SEP enhance pelvic nerve-stimulated and VIP-induced increases in vaginal and clitoral blood flow. It is also known that SEP inhibitors enhance VIP and nerve-mediated relaxations of the isolated vagina wall. Thus the present invention is advantageous as it helps provide a means for restoring a normal sexual arousal response-namely increased genital blood flow leading to vaginal, clitoral and labial engorgement. This will result in increased vaginal lubrication via plasma transudation, increased vaginal compliance and increased genital sensitivity. Hence, the present invention provides a means to restore, or potentiate, the normal sexual arousal response. By female genitalia herein it is meant: “The genital organs consist of an internal and external group. The internal organs are situated within the pelvis and consist of ovaries, the uterine tubes, uterus and the vagina. The external organs are superficial to the urogenital diaphragm and below the pelvic arch. They comprise the mons pubis, the labia majora and minora pudendi, the clitoris, the vestibule, the bulb of the vestibule, and the greater vestibular glands” (Gray's Anatomy, C. D. Clemente, 13th American Edition). R. J. Levin teaches that, because “ . . . male and female genitalia develop embryologically from the common tissue anlagen, [that] male and female genital structures are argued to be homologues of one another. Thus the clitoris is the penile homologue and the labia homologues of the scrotal sac . . . . ” (Levin, R. J. (1991), Exp. Clin. Efzdocrinol., 98, 6169).

With regard to MSD, in particular to MED, penile erection is a haemodynamic event which is dependent upon the balance of contraction and relaxation of the corpus cavernosal smooth muscle and vasculature of the penis (see Lerner, S. E. et al (1993). A review of erectile dysfunction: new insights and more questions. J. Urology 149: 1246-1255). Corpus cavernosal smooth muscle is also referred to herein as corporal smooth muscle or in the plural sense corpus cavernosa. Relaxation of the corpus cavernosal smooth muscle leads to an increased blood flow into the trabecular spaces of the corpus cavernosa, causing them to expand against the surrounding tunica and compress the draining veins. This produces a vast elevation in cavernosal blood pressure which results in an erection (see Naylor, A. M. (1998). Endogenous neurotransmitters mediating penile erection. Br. J. Urology 81: 424-431), hereinafter cited as “Naylor, 1998”). The changes that occur during the erectile process are complex and require a high degree of coordinated control involving the peripheral and central nervous systems, and the endocrine system (Naylor, 1998). Corporal smooth muscle contraction is modulated by sympathetic noradrenergic innervation via activation of postsynaptic α-adrenoceptors. MED may be associated with an increase in the endogenous smooth muscle tone of the corpus cavernosum. However, the process of corporal smooth muscle relaxation is mediated partly by non-adrenergic, non-cholinergic (=NANC) neurotransmission. There are a number of other NANC neurotransmitters found in the penis, other than nitric oxide (=NO), such as calcitonin gene related peptide (=CGRP) and VIP. The main relaxing factor responsible for mediating this relaxation is NO, which is synthesised from L-arginine by nitric oxide synthase (=NOS) (see e.g. Taub, H. C. et al (1993). Relationship between contraction and relaxation in human and rabbit corpus cavernosum. Urology 42: 698-704). It is thought that reducing corporal smooth muscle tone may aid NO to induce relaxation of the corpus cavernosum. During sexual arousal in the male, NO is released from neurones and the endothelium and binds to and activates soluble guanylate cyclase (sGC) located in the smooth muscle cells and endothelium, leading to an elevation in intracellular cyclic guanosine 3′,5′-monophosphate (cGMP) levels. This rise in cGMP leads to a relaxation of the corpus cavernosum due to a reduction in the intracellular calcium concentration ([Ca2+]i), via unknown mechanisms thought to involve protein kinase G activation (possibly due to activation of Ca²⁺ pumps and Ca²⁺-activated K⁺-channels).

Recently it has been shown that C-type natriuretic peptide (═CNP) may also play a role in MED, acting at the membrane-bound guanylyl cyclase B (=GC-B) which is expressed in human corpus cavernosum tissue. Stimulation of GC-B leads to an increase in intracellular cGMP and, consequently, smooth muscle relaxation. PDE5-inhibitors, e.g. sildenafil increase intracellular cGMP in corpus cavernosum tissue by inhibiting its breakdown. PDE5-inhibitors are inactive in the absence of a stimulator of cGMP formation, e.g. in the absence of NO. This finding suggests that the basal (unstimulated) rate of cGMP formation in the corpus cavernosum is rather low, so that inhibition of cGMP breakdown by PDE5 inhibitors is not sufficient for an erectile response without concomittant stimulation of guanylyl cyclase. Increasing the concentration of CNP leads to elevated intracellular cGMP concentration, by an increase in cGMP formation. Consequently, elevating the CNP concentration in the corpus cavernosum will presumably have similar effects as inhibiting PDE5. Due to their different mechanisms of action, i.e. increasing formation of cGMP vs. inhibition of its breakdown, the approaches of inhibiting PDE5 or the breakdown of CNP, respectively are deemed to be additive thus making it a reasonable assumption that a combination of these two mechanisms of action will be particularly effective in patients who do not respond to the administration of PDE5 inhibitors alone.

VIP positive nerve fibres have been found in the trabecular meshwork of the corpus cavernosum, suggesting a role of VIP release in penile erection. Effects of VIP are thought to be mediated via increases in cAMP and are thus complementary to those of cGMP-elevating agents. In patients with ED an intracavernosal injection of VIP (combined with the α-adrenoceptor antagonist phentolamine) was found to be a safe and effective treatment, with a response rate of 67% (erections sufficient for sexual intercourse).

The endopeptidases NEP and hSEP both degrade CNP and VIP and thereby limit the effects of CNP and VIP on cavernosal smooth muscle. Inhibition of CNP and VIP breakdown will lead to increased availability of these vasorelaxing factors thereby increasing blood flow to the corpus cavernosum which finally should result in improved erectile function. Support can be found for this from experimental data in rabbits, showing a significant increase in intracavernosal pressure and female genital blood flow after application of an NEP-inhibitor (see document WO 02/079143). Furthermore, a gene (SMR1) encoding a pro-peptide of the endogenous NEP-inhibitor sialorphine was found (see User H. M., Zelner D. J., McKenna K. E., McVary K. T. (2003). Microarray analysis and description of SMR1 gene in rat penis in a post-radical prostatectomy model of erectile dysfunction. J Urol.; 170(1):298-301) to be markedly downregulated (>80-fold) in a rat model of neurogenic erectile dysfunction suggesting that in this disease NEP activity may be enhanced and contribute to the development of erectile dysfunction.

Pharmacological Test Methods

1. Inhibition of Enzymatic Breakdown of CNP and VIP by the Test Compounds

The inhibition of the enzymatic breakdown of CNP and VIP by the test compounds of Formula I according to the invention was measured in an enzymatic in vitro assay according to the following protocol:

Enzymes:

a) hSEP (sol hu)(his)₆; or: His6-tagged hSEP ectodomain.
   stock solution: 53 μg/ml in 20 mM HEPES pH 7.2, 5% glycerol, 0.005% Tween20, 100 mM NaCl, purity>99%
   working solution: stock solution diluted with assay buffer to 5 μg/ml
   Supplier: Innogenetics, Ghent, Belgium. Preparation and purification of the protein were performed as described in WO 02/094176.
b) NEP (prepared from pig kidney cortex)
   stock solution: 120 μg/ml in 20 mM bisTris, purity>95%
   working solution: stock solution diluted with assay buffer to 5 μg/ml
   Supplier: Dr. Philippe Crine, Univ. of Montreal, Canada
      Substrates:
a) VIP
b) CNP (32-53)
   stock solution: 100 μM in assay buffer
   Supplier: Bachem, Weil am Rhein, Germany

Assay buffer: 100 mM Tris pH 7.0, 250 mM NaCl

All test compounds were dissolved in DMSO at 10 mM and further diluted with assay buffer.

Activity Assay Procedure

80 μl of assay buffer, 10 μl of enzyme working solution (NEP or hSEP) and 10 μl of peptide stock solution (VIP or CNP) were mixed in an Eppendorf vial and incubated for 120 min. at 37° C. The enzymatic reaction was subsequently terminated by heating to 95° C. for 5 min. After centrifugation (Heraeus Biofuge B, 3 min) the supernatant was subjected to HPLC.

Inhibition Assay Procedure

70 μl of assay buffer, 10 μl of enzyme working solution (NEP or hSEP) and 10 μl of a test compound solution were mixed in an Eppendorf vial and preincubated at 37° C. for 15 minutes. Then, 10 μl of peptide stock solution (VIP or CNP) was added and the reaction mixture was incubated at 37° C. for 60 min. to allow enzymatic hydrolysis. The enzymatic reaction was subsequently terminated by heating to 95° C. for 5 min. After centrifugation (Heraeus Biofuge B, 3 min) the supernatant was subjected to HPLC.

HPLC Procedure

For separating the remaining substrate from the cleavage products, a reversed phase HPLC technique with a CC 125/4 Nucleosil 300/5 C₁₈ RP column and a CC 8/4 Nucleosil 100/5 C18 precolumn (Macherey-Nagel, Duren, Germany) was used. 60 μl of the reaction samples were injected into the HPLC and the column was eluted at a flow rate of 1 ml/min with the following gradient:

| | | |
|---|---|---|
| Solution A: | 100% | H₂O + 0.5 M H₃PO₄ pH 2.0 |
| Solution B: | 100% | acetonitrile + 0.5 M H₃PO₄ |
| 0-2 min: | 5% | B |
| 2-7 min: | 5-50% | B |
| 7-8 min: | 50-90% | B |
| 8-10 min: | 90% | B |
| 10-12 min: | 90-5% | B |

All peptides were detected by absorbance at 214 nm (UV spectroscopy).

The percentage (=%) of hydrolysis was calculated on the basis of the peak area of the undegraded peptide for an enzyme containing sample Y in correlation to a sample containing the same concentration of peptide without enzyme (blank) by the following equation:
% hydrolysis=100*(blank−Y)

Basis of the calculation of % inhibition is the peak area of the undegraded peptide (VIP or CNP) for an inhibitor containing sample X in comparison to samples containing only peptide (blank) or peptide and enzyme without inhibitor (control) according to the following equation:
% inhib=100*(X−control)/(blank−control)

All samples were run in duplicate and mean values were used. A solvent control (0.1% DMSO) was added to each assay run.

CNP and VIP were cleaved by NEP and hSEP in vitro. Breakdown of both peptides was faster with hSEP than with NEP, as is shown in the following Table 1:

TABLE 1
Breakdown rates of VIP and CNP by NEP or hSEP

| | breakdown of CNP | | breakdown of VIP | |
|---|---|---|---|---|
| | hSEP | NEP | hSEP | NEP |
| degradation at t = 2 h | 46% | 39% | 36% | 28% |

The test compounds according to the invention were able to prevent degradation of CNP and VIP by both NEP and SEP. The respective IC₅₀-values for inhibition of breakdown are given below in table 2. Further, the compound thiorphan has also been tested as a reference standard for selective inhibition of NEP.

TABLE 2
Prevention of degradation of CNP and VIP by the test compounds

| | breakdown of CNP | | breakdown of VIP | |
|---|---|---|---|---|
| inhibition of | hSEP | NEP | hSEP | NEP |
| breakdown by | IC₅₀ (nM) | IC₅₀ (nM) | IC₅₀ (nM) | IC₅₀ (nM) |
| compound of | 0.7 | 7.5 | 3.5 | 3.3 |
| Formula II | | | | |
| compound 2 | 2.8 | 2.3 | 4.0 | 1.6 |
| compound of | 2.2 | 6.6 | 2.9 | 4.2 |
| Formula III | | | | |
| thiorphan | >1000 | 8.4 | >1000 | 6.4 |
| (standard) | | | | |

compound of Formula II: (3S,2′R)-3-[1-(2′-carboxy-4′-phenyl-butyl]-cyclopentan-1-carbonylamino]-2,3,4,5-tetrahydro-2-oxo-1H-1-benzazepin-1-acetic acid (compound of Formula Ia, R¹=hydrogen, R²=ethyl, R³=phenylethyl).

compound 2: (3S)-(3-{[1-(Benzyloxy-ethoxy-phosphorylmethyl)-cyclopentanecarbonyl]-amino}-2-oxo-2,3,4,5-tetrahydro-benzo[b]azepin-1-yl)-acetic acid tert-butyl ester (compound of Formula Ib, R¹=tert. butyl, R⁴=ethyl, R⁵=benzyl)

compound of Formula III: {(3S)-3-[({1-[(2R)-2-(ethoxycarbonyl)-4-(1-naphthyl)butyl]cyclopentyl}carbonyl)amino]-2-oxo-2,3,4,5-tetrahydro-1H-1-benzazepin-1-yl}acetic acid (compound of Formula Ia, R¹=hydrogen, R²=ethyl, R³=1-naphthylethyl)

2. Influence of Test Compounds on Big-ET-Induced Contraction of Human Cavernosal Strips In Vitro

Human erectile tissues were obtained from the corpus cavernosum of 9 patients undergoing surgery for implantation of penile prosthesis as treatment of erectile dysfunction or penile congenital curvature. After surgery, the tissue samples were transported and stored at 4° C. in RPMI-1640 medium containing HEPES (23.8 mM), penicillin (100 IU/ml) and streptomycin (0.1 mg/ml) until use (within 24 hours). Eight sections of 4×2×2 mm were excised from the corpus cavernosum of each donor for each experiment. Cavernosal strips were suspended in 5 ml organ chambers filled with Krebs-HEPES buffer. Organ chambers were maintained at 37° C. and continuously bubbled with 95% O₂ and 5% CO₂ to maintain a pH at 7.4. The cavernosal strips were connected to force transducers for isometric tension recording (Pioden controls Ltd, UK), and an initial tension of 1 g was applied. Following amplification, the tension changes were digitalized via a Mac Lab™/8 using Chart software (AD Instruments Ltd). The tissue preparation were allowed to equilibrate for 90 minutes, while being washed periodically (every 15 minutes) with fresh Krebs-HEPES buffer. When a stable resting tension was attained, the strips were set to at least 500 mg of basal tension. Following the equilibration period, the erectile tissues were primed with KCl 80 mM during 10 min. Once this priming period was achieved, the strips were washed by fresh Krebs-HEPES solution and allowed to re-equilibrate for 30 minutes at 500 mg of tension.

Once the strips were stabilized after the priming period, they were incubated with one of the following compounds (all at 10 μM, unless indicated otherwise) during 20 min: compound of Formula IV, compound of Formula V, SM-19712 (a selective and potent endothelin converting enzyme-1 inhibitor), DL-thiorphan (a selective and potent NEP inhibitor), and the combination of SM-19712, 10 μM and DL-thiorphan, and the common vehicle (phosphate buffer).

The strips were then exposed to the half-maximally effective concentration of big-ET-1 (10⁻⁸ M) determined in the preliminary experiments until a stable response was obtained.

Contractile responses were expressed as absolute change in maximal developed tension (in mg). To reduce the potential heterogeneity of responses between strips, the contraction to big-ET-1 was normalized by the contractile responses to KCl 80 mM obtained during the priming period.

TABLE 3
Effects of test compounds on maximal
big-ET-induced contraction of human corpus cavernosum

| | Maximal big-ET- | | |
|---|---|---|---|
| Compound | induced contraction | SEM | n |
| Vehicle | 149.2 | 3.96 | 6 |
| SM-19712 | 85.10 | 14.78 | 6 |
| Thiorphan | 91.57 | 13.61 | 6 |
| SM-19712 + Thiorphan | 21.28 | 6.26 | 6 |
| Cpd. of Formula IV | 32.80 | 5.20 | 5 |
| cpd. of Formula V | 49.60 | 10.92 | 6 |

The results are from duplicate or triplicate determinations from a single corpus cavernosum sample were averaged and values are expressed as percentage of the contractile responses induced by 80 mM of KCl, as mean □SEM, for n corpus cavernosum samples.

As indicated above, the compounds of Formula I also exert a certain ECE inhibitory share at their action profile. ET-1 induces a potent, slowly developing, and long-lasting contraction of penile arteries and the corpus cavernosum and may, thereby, contribute to keeping the penis in a flaccid state (see Andersson, K. -E. (2003) Erectile physiological and pathophysiological pathways involved in erectile dysfuncton. J Urol. 170:S1-S6). Recently, it has been shown that plasma levels of ET-1 are elevated in patients with erectile dysfunction compared to healthy controls. Moreover, ET-1 levels were the best independent predictors of erectile dysfunction in men without cardiovascular risk factors (see Bocchio M. et al. (2004), Endothelial cell activation in men with erectile dysfunction without cardiovascular risk factors and overt vascular damage. J Urol. 171:1601-1604). Based on these findings it can thus be deduced that inhibition of the endothelin pathway should improve human erectile function. Changes in the balance between contractant factors such as ET-1, and relaxant factors such as NO, VIP and CNP, will result in consecutive changes in penile blood flow and, hence, erectile function. Therefore, a compound that is able to reduce ET-1 formation, e.g. by inhibiting the endothelin-converting enzyme and simultaneously increase plasma concentrations of VIP and CNP, e.g. by inhibiting their breakdown by NEP and hSEP, can be expected to markedly increase blood flow to the corpus cavernosum and lead to improved erectile function.

In a fourth aspect the present invention also provides a pharmaceutical composition containing the dually acting inhibitors of NEP and of hSEP for the treatment of sexual dysfunction.

In a fifth aspect the present invention provides a pharmaceutical combination composition for the prophylaxis or treatment of SD, said pharmaceutical composition comprising pharmacologically effective quantities of each of
a) at least one neutral endopeptidase inhibitor,
b) at least one inhibitor of human soluble endopeptidase and
c) at least one compound supportive to the inventive use which is selected from the group consisting of the specific compounds or subgroups of PDE5 inhibitors, NPY receptor antagonists, PDE2 inhibitors, nitric oxide donors, dopamine receptor agonists, melanocortin receptor agonists, potassium channel openers, calcium activated potassium channel openers, α1-adrenoceptor antagonists, VIP receptor agonists or VIP analogues, α2-adrenoceptor antagonists, estrogens, medroxyprogesterone, medroxyprogesterone acetate, oestrogens, methyl testosterone hormone replacement therapy agents, testosterone, testosterone replacement agents, testosterone/oestradiol agents, estrogen agonists, serotonin receptor agonists or antagonists, prostanoid receptor agonists, purinergic receptor agonists, ECE inhibitors, endothelin receptor antagonists, NEP inhibitors and antidepressant agents.

In a preferred embodiment of said fifth aspect, a dually acting compound capable of inhibiting NEP and hSEP as subcombination of at least one neutral endopeptidase inhibitor (a) and at least one inhibitor of human soluble endopeptidase (b) can be used. Most preferred in this regard is the use of a compound of Formula I, more particular the use of a compound of Formula Ia.

The pharmaceutical combination compositions of the present invention comprise as component c) one or more other pharmaceutically active agents supportive to the inventive use, such as a NEP inhibitor, one or more of a PDE5 inhibitor (e.g. sildenafil, vardenafil and/or IC351), one or more of NPY receptor antagonist, one or more of a PDE2 inhibitor, one or more of a nitric oxide (NO) donor (e.g. NMI-921), one or more of a dopamine receptor agonist (e.g. apomorphine, Uprima, Ixsene), one or more of a melanocortin receptor agonist (e.g. Melanotan II or PT14), one or more of a potassium channel opener (e.g. a KATP channel opener (e.g. minoxidil, nicorandil) and/or a calcium activated potassium channel opener (e.g. BMS204352), one or more of an α-adrenoceptor antagonist (e.g. phentolamine, Vasofem, Vasomax), one or more of a VIP receptor agonist or a VIP analogue (e.g. Ro-125-1553) or VIP fragments, one or more of a α-adrenoceptor antagonist with VIP combination (e.g. Invicorp, Aviptadil), one or more of a α2-adrenoceptor antagonist (e.g. yohimbine), one or more of an estrogen, estrogen and medroxyprogesterone or medroxyprogesterone acetate (MPA) or oestrogen and methyl testosterone hormone replacement therapy (=HRT) agent (e.g. HRT especially Premarin, Cenestin, Oestrofeminal, Equin, Estrace, Estrofem, Elleste Solo, Estring, Eastraderm, Eastraderm TTS, Eastraderm Matrix, Dermestril, Premphase, Prempro, Prempak, Premique, Estratest, Estratest HS, Tibolone), one or more of a testosterone replacement agent (including DHEA (dehydroandrostendione), testosterone (Tostrelle) or a testosterone implant), one or more of a testosterone/oestradiol agent, one or more of an estrogen agonist (e.g. Lasofoxifene), one or more of a serotonin receptor agonist or antagonist (e.g. 5HT_{1A}, 5HT_{2C}, 5HT_{2A} and 5HT₃ receptor agonists and antagonists; as described in WO 00/28993), one or more of a prostanoid receptor agonist (e.g. Muse, alprostadil, misoprostol), one or more of a purinergic receptor agonist (especially P2Y2 and P2Y4), one or more of an ECE inhibitor, one or more of an endothelin receptor antagonists, or one or more antidepressant agents (e.g. bupropion, mirtazapine, nefazodone). Preferred components c) are PDE5 inhibitors.

In particular for human therapy, even though the compounds of the present invention can be administered alone, they will frequently be administered in admixture with a pharmaceutical carrier, excipient or diluent selected with regard to the intended route of administration and standard pharmaceutical practice. The components a), b) and c) of the pharmaceutical combination compositions can be administered jointly or consecutively in either order. By way of example, in the pharmaceutical compositions or the pharmaceutical combination compositions of the present invention, the agents of the present invention may be admixed with any suitable binder(s), lubricant(s), suspending agent(s), coating agent(s), or solubilising agent(s).

Where appropriate, the pharmaceutical compositions or the pharmaceutical combination compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the pharmaceutical compositions or the pharmaceutical combination compositions may be best used in the form of a sterile aqueous solution which may contain other substances, for example enough salts or monosaccharides to make the solution isotonic with blood. For buccal or sublingual administration the pharmaceutical compositions or the pharmaceutical combination compositions may be administered in the form of tablets or lozenges which can be formulated in a conventional manner. For oral, parenteral, buccal and sublingual administration to subjects (such as patients), the daily dosage level of the agents of the present invention may typically be from 10 to 500 mg (in single or divided doses).

Thus, and by way of example, tablets or capsules may contain from 5 to 600 mg of active agent for administration singly, or two or more at a time, as appropriate.

It is also possible to administer the agents of the present invention in sustained release formulations. In some applications, generally in humans, oral administration of the agents of the present invention is the preferred route, being the most convenient and can in some cases avoid disadvantages associated with other routes of administration-such as those associated with intracavemosal (i. c.) administration.

In circumstances where the recipient suffers from a swallowing disorder or from impairment of drug absorption after oral administration, the drug may be administered parenterally.

However, as with human treatments, it may be possible to administer the agent alone for veterinary treatments. Typically, the pharmaceutical compositions—which may be for human or animal usage will comprise any one or more of a pharmaceutically acceptable diluent, carrier or excipient. For veterinary use, the agent of the present invention is typically administered as a suitably acceptable formulation in accordance with normal veterinary practice and the veterinary surgeon will determine the dosing regimen and route of administration which will be most appropriate for a particular animal.

The choice of pharmaceutical carrier, excipient or diluent can be selected with regard to the intended route of administration and standard pharmaceutical practice.

As indicated above, the pharmaceutical compositions may comprise as, or in addition to the carrier, excipient or diluent any suitable binder(s), lubricant(s), suspending agent(s), coating agent(s) or solubilising agent(s).

In a sixth aspect, the invention also comprises a kit, comprising in separate containers in a single package pharmaceutical compositions for use in combination which comprises,
a) in one separate container a pharmaceutical composition comprising at least one neutral endopeptidase inhibitor and in a second separate container a pharmaceutical composition comprising at least one inhibitor of human soluble endopeptidase, or
b) in one separate container a pharmaceutical composition comprising a dually acting compound capable of inhibiting neutral endopeptidase and human soluble endopeptidase, and
c) in another separate container a pharmaceutical composition comprising at least one compound supportive to the inventive use which is selected from the specific compounds or subgroups of PDE5 inhibitors, NPY receptor antagonists, PDE2 inhibitors, nitric oxide donors, dopamine receptor agonists, melanocortin receptor agonists, potassium channel openers, calcium activated potassium channel openers, α1-adrenoceptor antagonists, VIP receptor agonists or VIP analogues, α2-adrenoceptor antagonists, estrogens, medroxyprogesterone, medroxyprogesterone acetate, oestrogens, methyl testosterone hormone replacement therapy agents, testosterone, testostrone replacement agents, testosterone/oestradiol agents, estrogen agonists, serotonin receptor agonists or antagonists, prostanoid receptor agonists, purinergic receptor agonists, ECE inhibitors, endothelin receptor antagonists, NEP inhibitors and antidepressant agents.

The following examples for pharmaceutical compositions according to the invention are set forth to illustrate the invention without limiting its scope in any aspect:

### EXAMPLE 1

### Tablets Containing Daglutril Tricalcium Phosphate Salt

Tablets were prepared with the following composition per tablet:

| | | |
|---|---|---|
| Daglutril tricalcium phosphate salt | 100 | mg |
| Corn starch | 60 | mg |
| Lactose | 55 | mg |
| Gelatine (as 10% solution) | 6 | mg |

The active substance, the corn starch and the lactose were thickened with the 10% gelatine solution. The paste was comminuted and the resulting granules were placed on a suitable sheet and dried at 45° C. The dried granules were fed through a crushing machine and mixed with the following further adjuvants in a mixer:

| | | |
|---|---|---|
| Talc | 5 | mg |
| Magnesium stearate | 5 | mg |
| Corn starch | 9 | mg |
| and then compressed to form tablets of 240 mg. | | |

### EXAMPLE 2

### Injection Solution Containing (3S,2′R)-3-[1-(2′-carboxy-4′-phenyl-butyl]-cyclopentan-1-carbonylamino]-2,3,4,5-tetrahydro-2-oxo-1H-1-benzazepin-1-acetic acid

An injection solution having the following composition per 5 ml was prepared:

| | | |
|---|---|---|
| (3S,2′R)-3-[1-(2′-carboxy-4′-phenyl-butyl)- | 10.00 | mg |
| cyclopentan-1- carbonylamino]-2,3,4,5- | | |
| tetrahydro-2-oxo-1H-1-benzazepin-1-acetic acid | | |
| Na₂HPO₄.7H₂O | 43.24 | mg |
| Na₂HPO₄.2H₂O | 7.72 | mg |
| NaCl | 30.00 | mg |
| Purified water | 4,948.00 | mg |

The solids were dissolved in water, the solution was sterilised and was poured into ampoules in portions of 5 ml each.

Further favourable pharmaceutical compositions of compounds according to the invention, in particular of compounds of Formula Ia, are known from e.g. documents WO 03/068266 A1 or WO 03/059939 A1.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of treating or inhibiting sexual dysfunction in a human or other mammal subject in need thereof, said method comprising administering to said subject a therapeutically effective amount of a dually acting compound capable of inhibiting neutral endopeptidase and human soluble endopeptidase.

2. A method according to claim 1, wherein said compound is a compound corresponding to Formula I 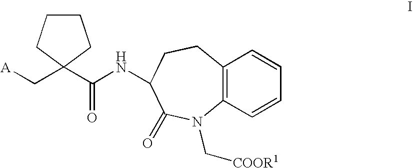
wherein
R¹ is hydrogen or a group forming a biolabile carbonic acid ester,
A represents a group selected from the subgroups a, 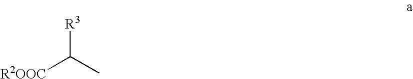
wherein
R² is hydrogen or a a group forming a biolabile carbonic acid ester and
R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group, and b, 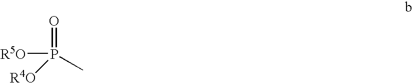
wherein
R⁴ is hydrogen or a group forming a biolabile phosphonic acid ester, and
R⁵ is hydrogen or a group forming a biolabile phosphonic acid ester,
or a physiologically compatible salt thereof.

3. A method according to claim 1, wherein said compound is a compound corresponding to Formula Ia, 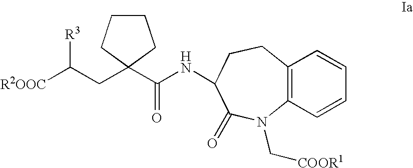
wherein
R¹ is hydrogen or a group forming a biolabile carbonic acid ester,
R² is hydrogen or a group forming a biolabile carbonic acid ester and
R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group,
or a physiologically compatible salt thereof.

4. A method according to claim 1, wherein said compound is selected from the group consisting of:
2-[1-(1-carboxymethyl-2-oxo-2,3,4,5-tetrahydro-1H-benzo[b]azepin-3-ylcarbamoyl)-cyclopentylmethyl]-4-phenyl-butyric acid ethyl ester;
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid ethyl ester;
2-[1-(1-carboxymethyl-2-oxo-2,3,4,5-tetrahydro-1H-benzo[b]azepin-3-ylcarbamoyl)-cyclopentylmethyl]-4-phenyl-butyric acid;
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid;
and physiologically compatible salts thereof.

5. A method according to claim 1, wherein the sexual dysfunction is female sexual dysfunction.

6. A method according to claim 1, wherein the sexual dysfunction is male sexual dysfunction.

7. A method according to claim 6, wherein the male sexual dysfunction is selected from the group consisting of erectile dysfunction, ejaculatory disorders and desire disorders.

8. A method according to claim 7, wherein the male sexual dysfunction is erectile dysfunction.

9. A pharmaceutical combination composition comprising pharmacologically effective quantities of:
a) at least one neutral endopeptidase inhibitor,
b) at least one inhibitor of human soluble endopeptidase, and
c) at least one compound selected from the group consisting of PDE5 inhibitors, NPY receptor antagonists, PDE2 inhibitors, nitric oxide donors, dopamine receptor agonists, melanocortin receptor agonists, potassium channel openers, calcium activated potassium channel openers, α1-adrenoceptor antagonists, VIP receptor agonists, VIP analogues, α2-adrenoceptor antagonists, estrogens, medroxyprogesterone, medroxyprogesterone acetate, methyl testosterone hormone replacement therapy agents, testosterone, testostrone replacement agents, testosterone/oestradiol agents, estrogen agonists, serotonin receptor agonists, serotonin receptor antagonists, prostanoid receptor agonists, purinergic receptor agonists, ECE inhibitors, endothelin receptor antagonists, NEP inhibitors and antidepressant agents.

10. A pharmaceutical composition according to claim 9, further comprising at least one pharmaceutically acceptable auxiliary or carrier.

11. A pharmaceutical composition according to claim 9, wherein said at least one neutral endopeptidase inhibitor (a) and said at least one inhibitor of human soluble endopeptidase (b) are present together in the form of a dually acting compound capable of inhibiting both neutral endopeptidase and human soluble endopeptidase.

12. A pharmaceutical composition according to claim 11, wherein said dually acting compound is a compound corresponding to Formula I 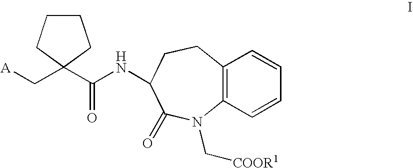
wherein
R¹ is hydrogen or a group forming a biolabile carbonic acid ester,
A represents a group selected from the subgroups a, 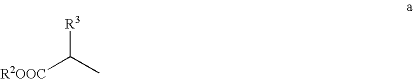
wherein
R² is hydrogen or a a group forming a biolabile carbonic acid ester and
R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group, and b, 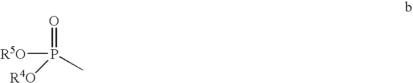
wherein
R⁴ is hydrogen or a group forming a biolabile phosphonic acid ester, and
R⁵ is hydrogen or a group forming a biolabile phosphonic acid ester,
or a physiologically compatible salt thereof.

13. A pharmaceutical composition according to claim 9, wherein component c) comprises at least one dopamine receptor agonist.

14. A pharmaceutical composition according to claim 13, wherein said at least one dopamine receptor agonist is selected from the group consisting of apomorphine, Uprima and Ixsene.

15. A kit comprising a plurality of separate containers in a single package, said containers containing pharmaceutical compositions for use in combination and comprising:
a1) in one separate container a pharmaceutical composition comprising at least one neutral endopeptidase inhibitor and in a second separate container a pharmaceutical composition comprising at least one inhibitor of human soluble endopeptidase, or
a2) in one separate container a pharmaceutical composition comprising a dually acting compound capable of inhibiting neutral endopeptidase and human soluble endopeptidase, and
b) in another separate container a pharmaceutical composition comprising at least one compound supportive to the inventive use which is selected from the group consiting of PDE5 inhibitors, NPY receptor antagonists, PDE2 inhibitors, nitric oxide donors, dopamine receptor agonists, melanocortin receptor agonists, potassium channel openers, calcium activated potassium channel openers, α1-adrenoceptor antagonists, VIP receptor agonists or VIP analogues, α2-adrenoceptor antagonists, estrogens, medroxyprogesterone, medroxyprogesterone acetate, methyl testosterone hormone replacement therapy agents, testosterone, testostrone replacement agents, testosterone/estradiol agents, estrogen agonists, serotonin receptor agonists, serotonin receptor antagonists, prostanoid receptor agonists, purinergic receptor agonists, ECE inhibitors, endothelin receptor antagonists, NEP inhibitors and antidepressant agents.

16. A method of treating or inhibiting sexual dysfunction in mammals and humans comprising administering to a subject in need thereof therapeutically effective amounts of
a) at least one neutral endopeptidase inhibitor,
b) at least one inhibitor of human soluble endopeptidase, and
c) at least one compound supportive to the inventive use which is selected from the group consisting of PDE5 inhibitors, NPY receptor antagonists, PDE2 inhibitors, nitric oxide donors, dopamine receptor agonists, melanocortin receptor agonists, potassium channel openers, calcium activated potassium channel openers, α1-adrenoceptor antagonists, VIP receptor agonists or VIP analogues, α2-adrenoceptor antagonists, estrogens, medroxyprogesterone, medroxyprogesterone acetate, methyl testosterone hormone replacement therapy agents, testosterone, testostrone replacement agents, testosterone/oestradiol agents, estrogen agonists, serotonin receptor agonists, serotonin receptor antagonists, prostanoid receptor agonists, purinergic receptor agonists, ECE inhibitors, endothelin receptor antagonists, NEP inhibitors and antidepressant agents.
